(19) United States
(12) Patent Application Publication
(10) Pub. No.: US 2026/0015591 A1
(43) Pub. Date: Jan. 15, 2026

(54) METHOD OF CULTURING WILD-TYPE HEPATITIS A VIRUS USING INDUCED DIFFERENTIATED PLURIPOTENT STEM CELLS DERIVED HEPATOCYTES

(71) Applicants: CHUNG ANG University Industry Academic Cooperation Foundation, Seoul (KR); Ministry of Food and Drug Safety, Cheongju-si (KR)

(72) Inventors: Changsun CHOI, Seoul (KR); Daseul YEO, Seoul (KR); Insun JOO, Cheongju-si (KR); Soon Han KIM, Busan (KR); Seung Hwan KIM, Cheongju-si (KR); Soo Hwan SUH, Cheongju-si (KR)

(73) Assignees: CHUNG ANG University Industry Academic Cooperation Foundation, Seoul (KR); Ministry of Food and Drug Safety, Cheongju-si (KR)

(21) Appl. No.: 19/267,321

(22) Filed: Jul. 11, 2025

(30) Foreign Application Priority Data
Jul. 15, 2024 (KR) 10-2024-0093250

(51) Int. Cl. C12N 7/00; C12N 5/071

(52) U.S. Cl. CPC C12N 7/00; C12N 5/067; C12N 2506/45; C12N 2770/32452

(57) ABSTRACT

A method of culturing wild-type hepatitis A virus (wtHAV) using induced differentiated pluripotent stem cell-derived hepatocytes. Specifically, iPSCs were used to differentiate into definitive endodermal (DE) cell, and then the DE cells were differentiated into four stages of hepatocyte progenitor (HpSC), hepatoblasts (HB), immature hepatocytes (immHep), and derived hepatocytes (dHep), and each stage cells were inoculated with wtHAV, and it was confirmed that all cells could be cultured more than the initial inoculation amount and the culture reproducibility. In addition, it was confirmed that the differentiated cells of each stage of the present invention can function as sufficient infection hosts, as the number of HAV gene copies increased after 3 days after inoculation. And thus, by inoculating HAV at each stage of differentiation and selecting the optimal time point of culture, it is possible to shorten the culture period and establish an efficient culture system with optimal effect.

### CROSS-REFERENCE TO RELATED APPLICATIONS

This application claims the benefit of priority under the Paris Convention from Korean Patent Application No. 10-2024-0093250, filed on Jul. 15, 2024. The contents of the above application are hereby incorporated by reference in their entirety.

### BACKGROUND OF THE INVENTION

### 1. Field of the Invention

The present invention relates to a method of culturing wild-type hepatitis A virus (wtHAV) using induced differentiated pluripotent stem cells, which can shorten the culture period and establish an efficient culture system by inoculating the wtHAV at each early stage (hepatic progenitor cell, hepatoblast) of hepatocyte differentiation and selecting the optimal time of culture.

### 2. Description of the Related Art

Induced pluripotent stem cells (iPSCs) refer to cells with pluripotency obtained by reverse differentiation from differentiated cells (e.g., somatic cells) and can differentiate into cells of various organs. Since iPS cells can be obtained by reprogramming differentiated cells by reverse differentiation inducing factors, it is possible to generate patient-immunocompatible pluripotent cell lines without somatic cell transfer. Because iPS cells can be derived from a patient's own cells, they can avoid immune rejection in clinical applications. In addition, iPS cells have the advantage of being free of bioethical controversy or religious criticism because they can be obtained without using eggs or embryos.

Since the first publication of the formation of iPSCs by reverse differentiation using mouse cells by Takahashi et al. in August 2006 (Takahashi, K., and Yamanaka, S. (2006). Induction of pluripotent stem cells from mouse embryonic and adult fibroblast cultures by defined factors. Cell 126, 663-676), Takahashi, K., et al. and Yu, J., et al. have reported the formation of iPSCs by reverse differentiation using human dermal fibroblasts (Takahashi, K., et al, (2007). Induction of pluripotent stem cells from adult human fibroblasts by defined factors. Cell 131, 861-872; Yu, J., et al. (2007). Induced Pluripotent Stem Cell Lines Derived from Human Somatic Cells. Science, New York, NY).

Hepatitis A is the most important cause of acute viral hepatitis in adults in Korea, accounting for more than 70% of all cases. Hepatitis A virus (HAV) is a member of the Picornaviridae family, is approximately 27 nm in size without an envelope, and has single-stranded RNA as its nucleic acid. HAV causes acute liver disease characterized by clinical symptoms such as fever, anorexia, nausea and vomiting, abdominal pain, dark urine, and jaundice after an incubation period of 28 days on average (SM Lemon et al. J. Hepatol, 68 (1): 167-184 (2018); Totsuka and Moritsugu, Intervirology, 42:63-68 (1999)). Surveys over the past decade have shown that the number of patients with hepatitis A has increased in the western United States, the Middle East, and some parts of Asia, raising concerns about the global spread of hepatitis A. In Korea, the number of people infected with hepatitis A has been rapidly increasing among young people in their teens and twenties who lack immunity (Nwachuku and Gerba, Rev. Environ. Contam. Toxicol. 186:1-56 (2006); Kim and Lee, Intervirology, 53 (1): 10-14 (2010); Korea Disease Control and Prevention Agency (KCDC), Infectious Disease Portal Legal Infectious Disease Statistics by Disease-Hepatitis A).

The main transmission route of HAV is the fecal-oral route, and it is transmitted through contaminated food or drinking water. The Advisory Committee on Immunization Practices (ACIP) recommends vaccination for travelers to and workers in areas with HAV endemicity, people with hepatitis B disease, people with chronic liver disease and chronic renal failure, and children living in areas with a high prevalence of hepatitis A.

Despite being a nationally mandated immunization, the hepatitis A vaccine frequently experiences shortages during outbreaks of the HAV.

Manufacturing a hepatitis A vaccine requires the production of HAV, but HAV replicates very slowly. Typically, viruses that infect humans can be cultured for as little as 2-3 days or as long as 7 days to replicate and isolate the virus, but in the case of HAV, it takes about a month of in vitro culture to obtain the virus. Even a period of one month is limited to cases if the virus is well adapted to cultured cells. Although certain cell lines susceptible to HAV infection—such as primary African green monkey kidney (AGMK) cells, FRhK-4, and BS-C-1—can support viral replication, they are unsuitable for efficient HAV production. These cell lines have not been characterized and their stability has not been verified as suitable for use as cell lines for human vaccine production. In particular, the cells in the example have been established as cell lines that can be cultured in vitro in flasks, etc., but they are classified as materials derived from rhesus macaques and savannah monkeys belonging to the long-tailed monkey family, which are internationally endangered species subject to restrictions by the CITES Convention, and are therefore difficult to import into Korea from ATCC (US), which sells cell lines commercially.

As a prior art of the present invention, Wheeler et al. (Wheeler, C. M., et al. (1986)) disclosed the culture of HAS-15 HAV in FRhK-4 cell line, Theresa et al. (Theresa, C. et al. (1989)) disclosed the culture of pHM-175 HAV in FRhK-4 cell line, and Morace et al. (Morace, G., et al. (1993)) disclosed the culture of HM175 cytopathic clonal HAV in Frp/3 cell line, but to date, the culture of wtHAV has not been established, and there is a need for a technology that can establish a cost-saving and efficient culture system.

Accordingly, iPSCs were used to differentiate into definitive endodermal cell (DE), and then the DE were differentiated into four stages of hepatocyte progenitor stem cells (HpSC), hepatoblasts (HB), immature hepatocytes (immHep), and derived hepatocytes (dHep), and each stage cells were inoculated with wtHAV, and it was confirmed that all cells could be cultured more than the initial inoculation amount. As a result of confirming the culture results at each differentiation stage according to the passage of time after wtHAV inoculation, especially when wtHAV was inoculated at the early stage of differentiation of iPSCs (HpSCs, HB) and differentiated into hepatocytes, it was confirmed that 5.70˜6.53 log₁₀/mL of wtHAV replicated in HpSCs and 5.47˜6.46 log₁₀/mL of wtHAV replicated in HB, and the reproducibility of the culture was also confirmed. In addition, the number of wtHAV genome copies number tended to decrease until the third day after inoculation, but the number of wtHAV genome copies number tended to increase after the third day, confirming that the hepatocytes differentiated from the iPSCs of the present invention can function as sufficient infection hosts for wtHAV culture. Therefore, while the conventional HAV culture method takes more than 4 weeks and cannot culture wtHAV, this culture method can shorten the culture period by selecting the optimal culture time point by inoculating wtHAV at each differentiation stage, and can establish an efficient culture system capable of culturing wtHAV in addition to cell-adapted HAV strain such as HM175.

### SUMMARY OF THE INVENTION

It is an object of the present invention to provide a method of culturing wtHAV using iPSCs-derived hepatocytes.

To achieve the above object, the present invention provides a method of culturing wtHAV using iPSCs-derived hepatocytes, comprising the following steps:
a) a step of inoculating wtHAV within a set period of time according to the cell differentiation stage before the iPSCs differentiate into derived hepatocytes (dHEP); and
b) a step of culturing for a set period of time according to the cell differentiation stage after inoculation in step a).

The method of culturing wtHAV using iPSC-derived hepatocytes of the present invention confirms that the wtHAV can be cultured in an amount more than the initial inoculation amount by inoculating wtHAV at each stage of hepatocyte differentiation, thereby shortening the culture period by selecting the optimal culture time for each differentiation stage, reducing costs, and establishing an efficient culture system with increased culture efficiency and a highly versatile HAV culture system.

In addition, since the culture method of the present invention can secure a desired amount of virus compared to existing culture methods, it has the advantage of increasing the efficiency of manufacturing wtHAV vaccine and reducing manufacturing costs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing the entire differentiation process from induced differentiated pluripotent stem cells (iPSCs) to hepatocytes.

FIG. 2 is a schematic diagram showing the culture process of iPSCs.

FIG. 3 is a schematic diagram showing the process of changing the differentiation medium during the process of differentiating HpSC into dHEP.

FIG. 4 is a schematic diagram showing the wtHAV culture inoculation and recovery times.

FIG. 5 is a schematic diagram showing the wtHAV culture experiment.

FIG. 6 presents a series of micrographs illustrating the cell morphology observed on the first day of culture of iPSCs undergoing differentiation.

FIG. 7 is a set of photographs showing the results of observing the cell morphology on the third day of culture of iPSCs.

FIG. 8. Immunofluorescence analysis of pluripotency markers in ChiPSC18 cells (#25). Representative micrographs (×40) show the expression of pluripotency markers SSEA4, OCT4, SOX2, and TRA-1-60, with nuclear counterstaining using DAPI.

FIG. 9 is a set of photographs showing the morphology of definitive endoderm cells (ChiPSC18) by day.

FIG. 10 is a set of photographs showing the morphology of endoderm cells (CMC-hiPSC-009) by day.

FIG. 11 is an Immunofluorescence analysis of the differentiation of endoderm cells in CMC-hiPSC-003 (#38) for SOX17.

FIG. 12 is an Immunofluorescence analysis of the differentiation of endoderm cells in CMC-hiPSC-011 (#44) for SOX17.

FIG. 13 is a set of photographs showing the results of observing the morphology of cells at each stage of differentiation.

FIG. 14 is an Immunofluorescence analysis of the differentiation of the cell activity of ChiPSC18 cells using the CYP3A4 matured hepatocyte marker.

FIG. 15A is a set of graphs showing the maximum amount of replication at each stage of differentiation of ChiPSC18 cells following inoculation with HAV.

FIG. 15B is a set of graphs showing the maximum amount of replication at each stage of differentiation of CMC-hiPSC-009 cells following inoculation with HAV.

FIGS. 16A to 16D are graphs showing the culture results at each stage of differentiation over time following inoculation with wtHAV. FIG. 16A shows the replication genome copy number of wtHAV (serum 1) in ChiPSC18-derived hepatic lineage cells. Quantification of HAV RNA genome equivalents (log 10 copy/mL) over 33 days post-infection (dpi) in cells at various differentiation stages: HpSC, HB, immHep, and dHep. Error bars represent standard deviations from biological replicates. FIG. 16B shows the replication genome copy number of wtHAV (serum 6) in ChiPSC18-derived hepatic lineage cells. Quantification of HAV RNA genome equivalents (log 10 copy/mL) over 33 dpi in cells at various differentiation stages: HpSC, HB, immHep, and dHep. Error bars represent standard deviations from biological replicates. FIG. 16C shows the replication genome copy number of wtHAV (serum 1) in CMC-hiPSC-009-derived hepatic lineage cells. Quantification of HAV RNA genome equivalents (log 10 copy/mL) over 30 dpi in cells at various differentiation stages: HpSC, HB, immHep, and dHep. Error bars represent standard deviations from biological replicates. FIG. 16D shows the replication genome copy number of wtHAV (serum 6) in CMC-hiPSC-009-derived hepatic lineage cells. Quantification of HAV RNA genome equivalents (log 10 copy/mL) over 30 dpi in cells at various differentiation stages: HpSC, HB, immHep, and dHep. Error bars represent standard deviations from biological replicates.

FIG. 17 is an Immunofluorescence assay of viral VP3 protein expression, visualized at 10× magnification with 20× magnification inset.

FIGS. 18A to 18D are graphs showing the culture results of HAV at each stage of differentiation according to HAV culture time. FIG. 18A shows the culture results over time after inoculation of cells in the HpSC differentiation stage with HAV, FIG. 18B shows the culture results over time after inoculation of cells in the HB differentiation stage with HAV, FIG. 18C shows the culture results over time after inoculation of cells in the immHep differentiation stage with HAV, and FIG. 18D shows the culture results over time after inoculation of cells in the dHep differentiation stage with HAV, respectively.

FIG. 19 is a graph showing the culture results by inoculating the HAV HM175 strain at each stage of hepatocyte differentiation.

### DETAILED DESCRIPTION OF THE INVENTION

Hereinafter, the present invention is described in detail.

The present invention provides a method of culturing wtHAV using iPSC-dHEP, comprising the following steps:
a) a step of inoculating wtHAV within a set period according to the cell differentiation stage before the iPSCs differentiate into dHEP; and
b) a step of culturing for a set period of time according to the cell differentiation stage after inoculation in step a).

In the present invention, the iPSCs may be hESCs, hiPSCs, disease-specific hiPSCs, GFP-tagged hiPSCs or gene-edited hiPSCs, preferably hESCs or hiPSCs, and most preferably hiPSCs.

In the present invention, the iPSCs are hESCs, and the hESCs may include SNUhES3, SNUhES4, SNUhES31 or CHA-hES15 cell line.

In the present invention, the iPSCs are hiPSCs, and the hiPSCs may include hFmiPS1, hFmiPS1, hFSiPSC3-1, hUSiPS2, hAdMSiPS1, CMC-hiPSC-003, CMC-hiPSC-005, CMC-hiPSC-009, CMC-hiPSC-011, CMC-hiPSC-022, NU01-EiPS07, ChiPSC18 or PB01-EiPS21 cell line, preferably ChiPSC18, CMC-hiPSC-003, CMC-hiPSC-005, CMC-hiPSC-009, CMC-hiPSC-011 or CMC-hiPSC-022, more preferably ChiPSC18, CMC-hiPSC-003, CMC-hiPSC-009 or CMC-hiPSC-011.

In the present invention, the cell differentiation stage may be a HpSC differentiation stage, HB differentiation stage, and an immHep differentiation stage, but not always limited thereto.

In the present invention, the period determined according to the cell differentiation stage of step a) may be 7 to 13 days, preferably 9 to 11 days, from the initial culture time point, which is the stage where the iPSCs differentiate into HpSC through DE.

In the present invention, the period determined according to the cell differentiation stage of step a) may be 13 to 18 days, preferably 14 to 16 days, from the initial culture time point, which is the stage where the iPSCs differentiate into HB through DE, and HpSC.

In the present invention, the period determined according to the cell differentiation stage of step a) may be 18 to 21 days from the initial culture time point, which is the stage where the iPSCs differentiate into immHep through DE, HpSC, and HB.

In the present invention, the inoculation of wtHAV in step a) may be inoculation of serum, blood, sputum, urine, or biological tissue taken from a patient infected with HAV, or food contaminated with HAV.

In the present invention, the inoculation amount of HAV in step a) may be 3.0 to 6.0 log₁₀/mL, preferably 3.5 to 5.5 log₁₀/mL, more preferably 3.5 to 5.0 log₁₀/mL.

In the present invention, the HAV may have a genotype of IA, IB, IIA, IIB, IIIA or IIIB, preferably a genotype of IA, IB, IIA or IIB, more preferably a genotype of IA.

In the present invention, when the wtHAV is inoculated at the HpSC differentiation stage, the set culture period of step b) may be 14 to 32 days after the inoculation, preferably 16 to 32 days.

In the present invention, when the wtHAV is inoculated at the HB differentiation stage, the set culture period of step b) may be 14 to 27 days after the inoculation, preferably 16 to 27 days.

In the present invention, when the wtHAV is inoculated at the immHep differentiation stage, the set culture period of step b) may be 12 to 23 days after the inoculation.

In the present invention, the culture of step b) may be performed in a 37° C., 5% CO₂ incubator for 30 minutes to 2 hours, preferably 45 minutes to 1 hour and 30 minutes after the virus inoculation in step a).

In the present invention, in the culture of step b), a medium comprising a differentiation signal molecule is added after recovery of the culture medium at each detection time point after virus inoculation. Hepatocyte maintenance medium is used as the medium containing the signal molecule.

In the present invention, the production amount of wtHAV by the culture method can be 1.1 to 2.0 times, preferably 1.1 to 1.9 times, more preferably 1.2 to 2.0 times the initial inoculation amount of wtHAV.

The present invention provides a hepatitis A vaccine composition comprising a viral agent, which is an inactivated culture supernatant containing viruses prepared by the method of culturing wtHAV, and a pharmaceutically acceptable carrier or excipient.

In the present invention, the vaccine composition can be used as a vaccine for preventing or treating hepatitis A.

In the present invention, the vaccine is an inactivated vaccine, and the agent used for producing the inactivated vaccine may be formaldehyde. Formaldehyde has the advantage of easily inactivating viruses and fixing viral proteins, which facilitates immune response in the administered subject.

The compositions of the present invention may also include carriers, diluents, excipients, or combinations of two or more thereof that are commonly used in biological preparations. The pharmaceutically acceptable carriers are not particularly limited as long as they are suitable for delivering the composition in vivo.

The composition of the present invention may further comprise pharmaceutically acceptable additives.

The therapeutic composition of the present invention can be administered parenterally (for example, intravenously, subcutaneously, intraperitoneally, or topically) or orally, depending on the intended method, and the dosage varies depending on the subject's body weight, age, gender, health condition, diet, administration time, administration method, excretion rate, and disease severity.

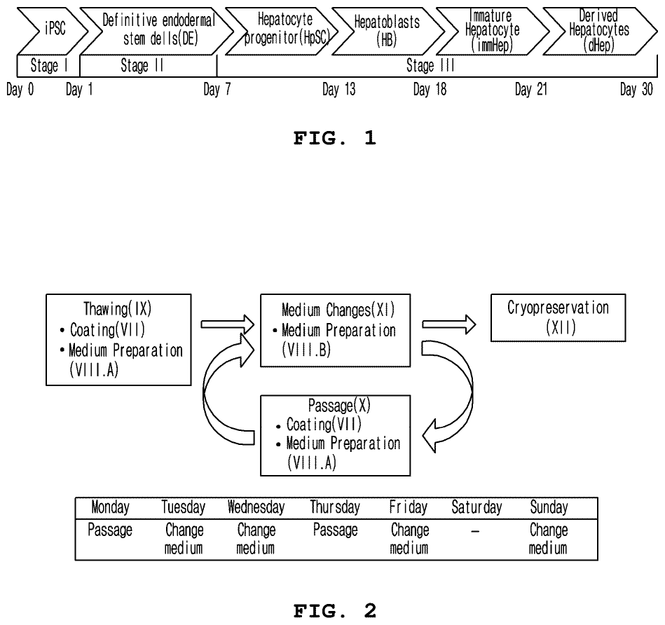

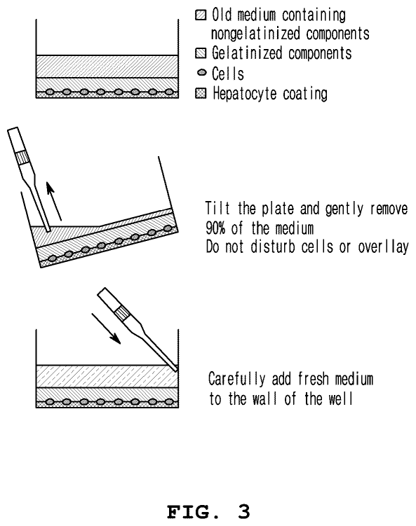

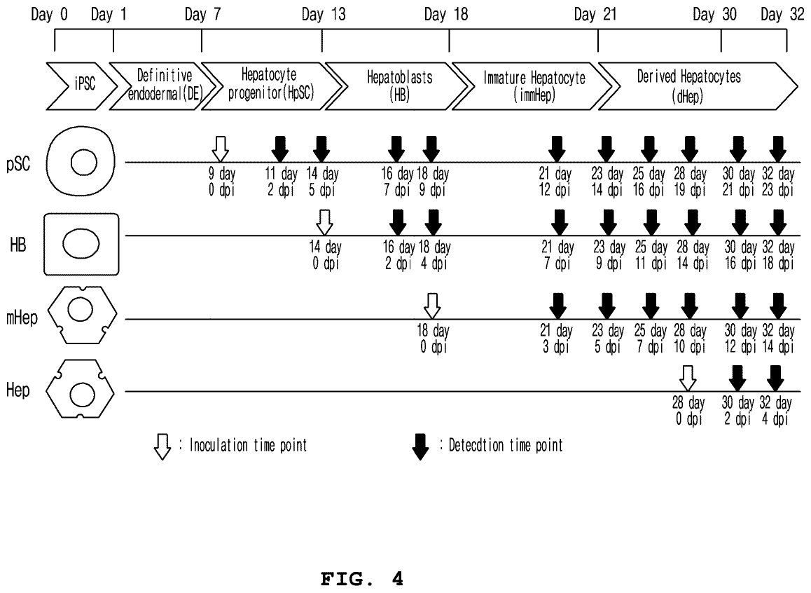

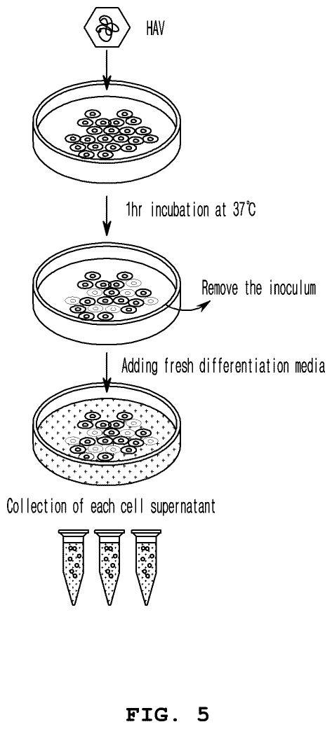

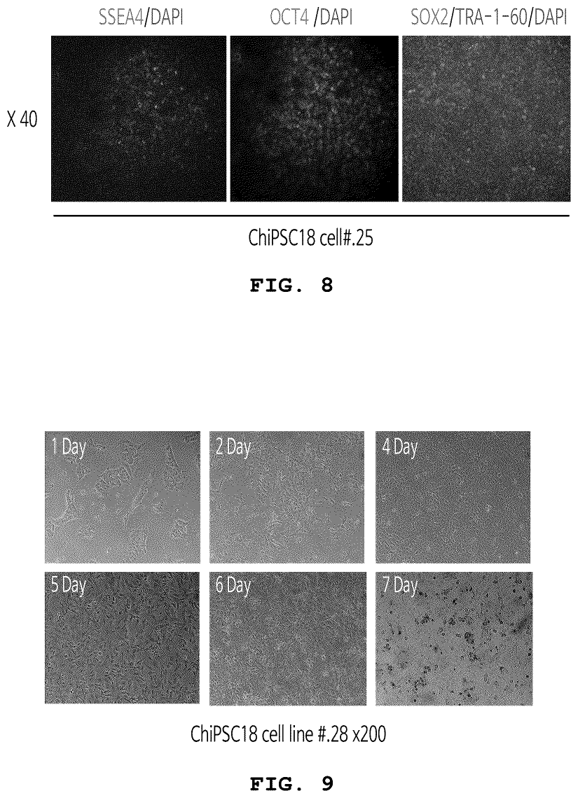

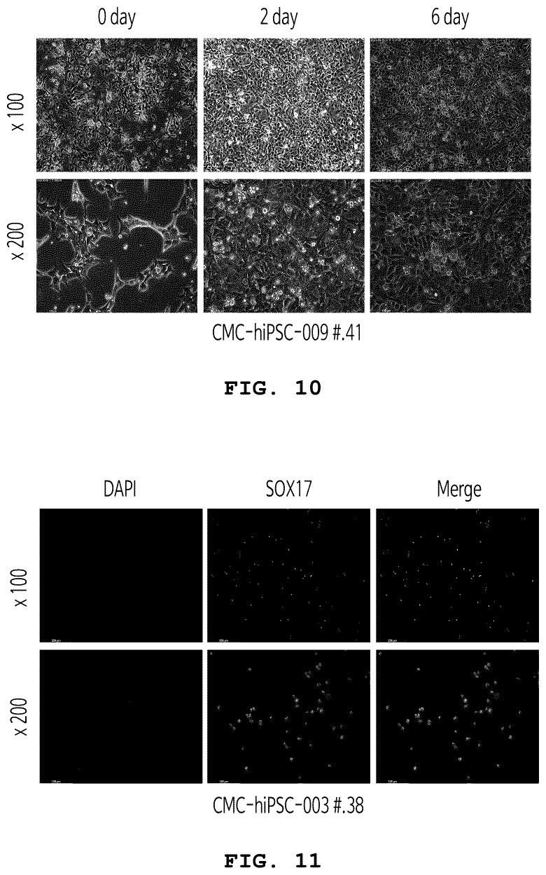

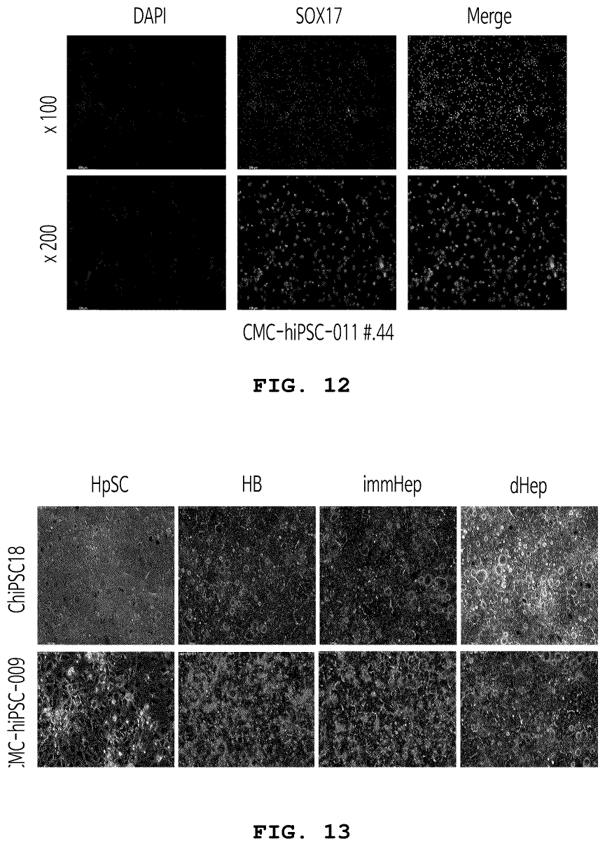

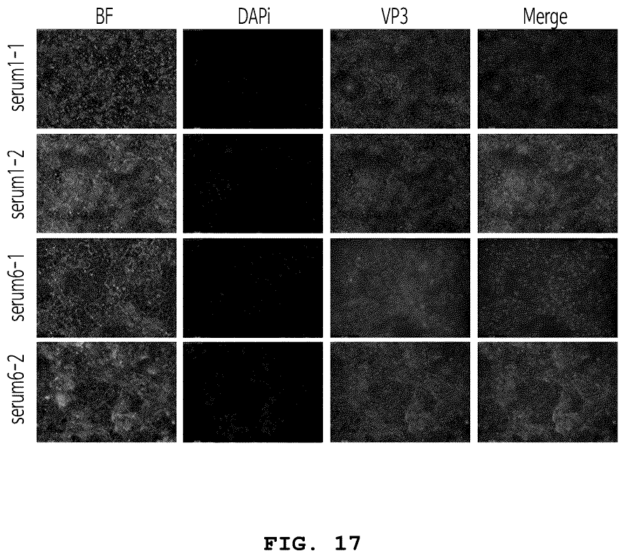

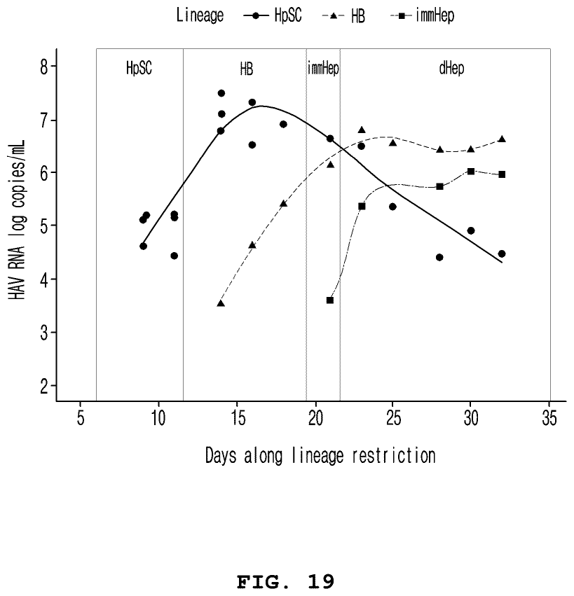

In a specific embodiment of the present invention, the present inventors have received or purchased four types of iPSCs according to Table 1. As a result of observing four types of cell morphology according to culture, it was confirmed that iPSC characteristics and undifferentiation ability (FIG. 8) appeared on day 3 (FIG. 7), although they were not present on day 1 (FIG. 6), and that an average culture period of 3 to 4 days was required. The entire culture process was carried out according to the schematic diagrams in FIGS. 1 and 2. The differentiation medium at each stage was carried out as shown in FIG. 3. The optimal culture time was explored by inoculating wtHAV at each hepatocyte differentiation stage—HpSC, HB, immHep, and dHep (FIG. 4)—and it was confirmed that all were cultured at levels exceeding the inoculation amount, and culture reproducibility was also confirmed (FIGS. 14 to 19). In addition, when HM175 strain was cultured according to the hepatocyte differentiation stages (FIG. 19), a tendency for the amount of virus replication to increase depending on the point of change in the differentiation stage was observed.

Through this, it was confirmed that the method of culturing wtHAV of the present invention can be used to inoculate HAV at each differentiation stage and select the optimal time of culture, thereby shortening the period of HAV culture, reducing costs, and establishing an effective culture system using the point of differentiation stage change.

Hereinafter, the present invention will be described in detail by the following examples. However, the following examples are only for illustrating the present invention, and the contents of the present invention are not limited thereto.

### Example 1: Obtaining Cell Lines for wtHAV Culture

A total of four types of iPSCs were used in the present invention. One type (ChiPSC18) was purchased from Cellartis (Takara Bio Europe), and three types (CMC-hiPSC-003, CMC-hiPSC-009, and CMC-hiPSC-011) were provided from the National Stem Cell Bank (Table 1).

TABLE 1

| | Species | Parental | | | |
|---|---|---|---|---|---|
| Cell line | of origin | Cell | Reprogram | QC | Index |
| ChiPSC18 | Human | Skin | Retrovirus | HLA- | Hepatocyte |
| | | fibroblast | | homozygote | differentiation |
| | | | | | ability confirmed |
| CMC- | Human | Bone | Sendai | HLA- | Hepatocyte |
| hiPSC-003 | | marrow | virus | homozygote | differentiation |
| | | blood | | | ability confirmed |
| CMC- | Human | Cord | Sendai | HLA- | Hepatocyte |
| hiPSC-009 | | blood | virus | homozygote | differentiation |
| | | | | | ability confirmed |
| CMC- | Human | Cord | Sendai | HLA- | Hepatocyte |
| hiPSC-011 | | blood | virus | homozygote | differentiation |
| | | | | | ability confirmed |

Immunofluorescence assay (IF) was used for the phenotype pluripotent analysis of the iPSCs provided. Information on the antibodies used is shown in Table 2.

TABLE 2

| Antibody | Role | Host | Clonality |
|---|---|---|---|
| OCT4 | Transcription factor that binds to octamer | Rabbit | Polyclonal |
| | motif (5′-ATTTGCAT-3′). | | |
| | Forms a trimeric complex with SOX2 or | | |
| | SOX15 in DNA and controls the | | |
| | expression of several genes involved in | | |
| | embryonic development, such as YES1, | | |
| | FGF4, UTF1, and ZFP206. | | |
| SSEA4 | A glycolipid epitope used as a marker for | Mouse | Polyclonal |
| | many human pluripotent cells. | | |
| SOX2 | Regulating embryonic development and | Rat | Polyclonal |
| | determining cell fate. | | |
| TRA-1-60 | A cell surface antigen co-expressed with | Mouse | Polyclonal |
| | SSEA-3, SSEA-4, and TRA-1-81 in human | | |
| | embryonic stem cells, embryonic | | |
| | carcinoma cells, and induced pluripotent | | |
| | stem cells. | | |

(iPSC undifferentiation ability marker)

The hepatocyte differentiation capability of each cell line was confirmed, and the differentiated hepatocytes possessed cytochrome P450 (CYP) activity and could be cultured for approximately 20 days after hepatocyte differentiation.

As a result of observing the morphology of four types of iPSCs, tight cellular packing and prominent nucleoli, which are characteristics of iPSCs, were confirmed on day 3 (FIG. 7), although they were not observed on day 1 (FIG. 6), indicating that an average culture period of 3 to 4 days was required.

As a result of IF assay using the undifferentiation markers OCT4, SSEA4, SOX2, and TRA-1-60, it was confirmed that undifferentiation was maintained by observing the green fluorescence of the target undifferentiation markers SSEA4, OCT4, and SOX2. In addition, the maintenance of undifferentiation ability was confirmed by observing the red fluorescence of TRA-1-60, and the undifferentiation ability was confirmed by observing the yellow fluorescence of the merged region of SOX2 and TRA-1-60 (FIG. 8).

### Example 2: Differentiation of Hepatocytes from iPSCs

The process of differentiation of hepatocytes from the iPSCs of Example 1 is shown in FIG. 1. In the present invention, iPSCs were cultured in a monolayer form using the feeder-free Cellartis DEF-CS™ culture system (Cellartis, Cat #. Y30010, Y30020), and then differentiated into hepatocytes using Hepatocyte Differentiation System (Cellatis, Cat #. Y30055).

<2-1> Culture of iPSCs

The iPSC culture was performed using the Cellartis DEF-CS™ culture system (Cellartis, Cat #. Y30010, Y30020), and the entire process of culture was as shown in the schematic diagram of FIG. 2. In this iPSC culture process, iPSC culture was performed in a monolayer form, in which the number of cells and the culture medium in relation to the area volume were the main factors, and single-cell culture was the main factor in maintaining undifferentiation ability. The method of culturing iPSCs is as follows.

### 1. Cell Culture Vessel Coating

4 mL of Cellartis DEF-CS COAT-1 solution was diluted 1:20 in D-PBS+/+ (PBS Dulbecco's with Ca²⁺& Mg²⁺). The diluted Cellartis DEF-CS COAT-1 solution was appropriately dispensed into cell culture flasks (0.1 mL/cm²). Cell culture flasks were reacted in an incubator for at least 20 minutes or at room temperature (15 to 25° C.) for 0.5 to 3 hours. After removing the Cellartis DEF-CS COAT-1 solution, the cells were ready for seeding.

### 2. Preparation of Cellartis DEF-CS Medium

An appropriate volume of thawing or passaging medium was prepared by adding DEF-CS GF-1 (dilution ratio 1:333), GF-2 (dilution ratio 1:1000), and GF-3 (dilution ratio 1:1000) to the Cellartis DEF-CS Basal medium. The prepared medium was stored at 4° C., and fresh medium was prepared in small portions according to the scheduled use.

An appropriate volume of DEF-CS medium for maintaining iPSC lines was prepared by adding GF-1 (dilution ratio 1:333) and GF-2 (dilution ratio 1:1000) to the Cellartis DEF-CS Basal medium. The prepared medium was stored at 4° C., and fresh medium was prepared in small portions according to the scheduled use.

### 3. Thawing iPS Cell Lines

When thawing iPSCs using the Cellartis DEF-CS culture system, approximately 1.5 to 2.5×10⁶ cells/cm² of cells and 0.3 to 0.4 mL medium/cm² of medium were used. The cell culture vessel was coated as in step 1 above, and the thawing medium was prepared as in step 2.

4 mL of the Cellartis DEF-CS medium was warmed at 37° C. Using forceps, the vials stored in liquid nitrogen were thawed in a 37±1° C. water bath. The cell suspension was added to the thawing medium prepared in step 2 above. After centrifugation at 300×g for 1 minute, the supernatant was aspirated, and the pellet was properly resuspended using the thawing medium (37±1° C.). The number of cells was counted using a haemocytometer or cell counter. A cell suspension was prepared with 1.5 to 2.5×10⁵ cells/cm² of cells in 0.3 to 0.4 mL medium/cm² of culture medium. After removing the COAT-1 solution from the cell culture vessel, cells were seeded and cultured in a 37±1° C., 5% CO₂ incubator.

### 4. Passaging iPS Cell Lines

The recommended cell seeding dose was 4.0 to 5.0×10⁴ cells/cm², and the recommended passaging cell dose was 1.5 to 3.0×10⁵ cells/cm². The cell culture vessel was coated as in step 1 above, and the thawing medium was prepared as in step 2.

Cell morphology was observed using a microscope. After removing the culture medium, cells were washed once with PBS Dulbecco's without Ca²⁺& Mg²⁺ (D-PBS−/−). Cells were detached by adding 20 μL/cm² TrypLE Select and reacting for 5-8 minutes in an incubator. A cell suspension was prepared using passaging medium. After centrifugation at 200×g for 2 to 5 minutes, the supernatant was aspirated, and the pellet was properly resuspended using the passaging medium. The number of cells was counted using a haemocytometer or cell counter. The COAT-1 solution was removed from the cell culture vessel. 0.15 to 0.25 mL medium/cm² of medium was added to 4.0 to 5.0×10⁴ cells/cm² of cells, and cultured in a 37±1° C., 5% CO₂ incubator.

### 5. Changing Medium

The culture medium was changed daily except on the day of passaging. If the medium color turned yellow due to increased metabolic activity when the medium was replaced, the volume of the culture medium was increased. The maintenance medium of step 2 was prepared and warmed to 37±1° C. Cell morphology was observed using a microscope. After carefully removing the culture medium, a fresh medium was carefully replaced on the walls of the cell culture vessel.

### 6. Cryopreserving iPS Cell Lines

The iPS cell lines were cryopreserved using STEM-CELLBANKER (Zenoaq Resource). When preserving, a freezing agent containing DMSO was used. 2.5 to 3.5×10⁶ cells/mL of cells were mixed with 2 mL of STEM-CELLBANKER. The cells were frozen by cooling 1° C. per second using a cell cooler at −80° C., and stored in liquid nitrogen after day 1. To maintain undifferentiation ability and prevent mixing with spontaneously differentiated cells, cryopreservation was performed with the following information:
cell line name
passaging number
number of cells
cryopreservation date
stock composition used for cryopreservation
experimenter's name.

### ≤2-2> Differentiation of DE

DE culture was performed using Cellartis iPS Cell to Hepatocyte Differentiation System (Cellartis, Cat #. Y30055). When seeding iPSCs for DE culture, if the seeding density is low relative to the area, the cells may die during the differentiation process, and if the seeding density is high relative to the area, the endoderm differentiation may not occur evenly, so caution is required. Therefore, the culture method according to the present invention is optimized for 2D culture, and 6-well plate culture is recommended. The materials used for endoderm culture are shown in Table 3, and the endoderm culture was performed using the following method.

TABLE 3

Materials in iPS Cell to Hepatocyte Differentiation System
1 tube Definitive Endoderm Differentiation Coating (10 mL)
1 bottle Definitive Endoderm Differentiation Day 0 (18 mL)
1 bottle Definitive Endoderm Differentiation Day 1 (18 mL)
1 bottle Definitive Endoderm Differentiation Day 2 (18 mL)
1 bottle Definitive Endoderm Differentiation Day 3 (25 mL)
1 bottle Definitive Endoderm Differentiation Day 4 (47 mL)
1 bottle Definitive Endoderm Differentiation Day 6 (47 mL)

Additional materials required for culture
Fetal bovine serum (FBS) or KnockOut Serum Replacement (KO-SR)
PBS Dulbecco's with Ca²⁺ & Mg²⁺ (D-PBS +/+)
PBS Dulbecco's w/o Ca²⁺ & Mg²⁺ (D-PBS −/−)
TrypLE Select Enzyme (1X), without phenol red
Equipment to check or control the temperature at 16-18° C., such as a water bath, a
Biosafety Cabinet with Temp-Zone area or an infrared thermometer.
96-well plates, flat bottom, cell-culture treated
24-well plates, flat bottom, cell-culture treated
Cell culture vessels, tissue-culture treated polystyrene surface
General cell culture equipment used in cell culture laboratory

### 1. Amount of Coating and Medium

The recommended amounts of coating and medium are shown in Table 4 below.

TABLE 4

| Format | coating | day 0, 1, 2 | day 3 | day 4, 6 |
|---|---|---|---|---|
| 6 well plate | 1 mL | 2 mL | 2.7 mL | 5 mL |
| T25 flask | 2.5 mL | 5 mL | 7 mL | 13 mL |
| T75 flask | 7.5 mL | 15 mL | 20 mL | 40 mL |

### 2. Day 0: Preparation of Endoderm Cell Differentiation

The coating solution was thawed and warmed at room temperature. Day 0 endoderm medium was warmed to 37° C.

The coating solution was dispensed into cell culture vessels (0.1 mL/cm²), which were incubated at room temperature for 30 to 120 minutes, and then the coating solution was removed to prepare for seeding. A iPSC suspension was prepared using TrypLE Select Enzyme, the number of iPSCs was counted, and an appropriate number of iPSCs were transferred to a 15 ml tube. The cell density should be 4.0×10⁴ cells/cm². The cell suspension was centrifuged at 200×g for 5 minutes at room temperature, and the supernatant was removed. The pellet was resuspended in the day 0 differentiation medium and seeded into coated cell culture vessels. The cell culture vessels were cultured in a 37° C., 5% CO₂ incubator.

### 3. Days 1-6: Change Endoderm Cell Medium

After warming the endoderm differentiation medium for each day at 37° C., the culture medium was removed using a pipette or vacuum pump. Then, an appropriate amount of the endoderm differentiation medium was added. The cell culture vessels were cultured in a 37° C., 5% CO₂ incubator.

### <2-3> Confirmation of Definitive Endodermal Stem Cell (DE) Differentiation

For qualitative analysis of endoderm differentiation, IF assay was used. The antibodies used herein are shown in Table 5.

TABLE 5

| Marker | Host | Catalog # |
|---|---|---|
| FOXA2 | Rabbit | ab108422 |
| SOX17 | Goat | R&D Systems #AF1924 |

### (List of Definitive Endodermal Cell Marker Antibodies)

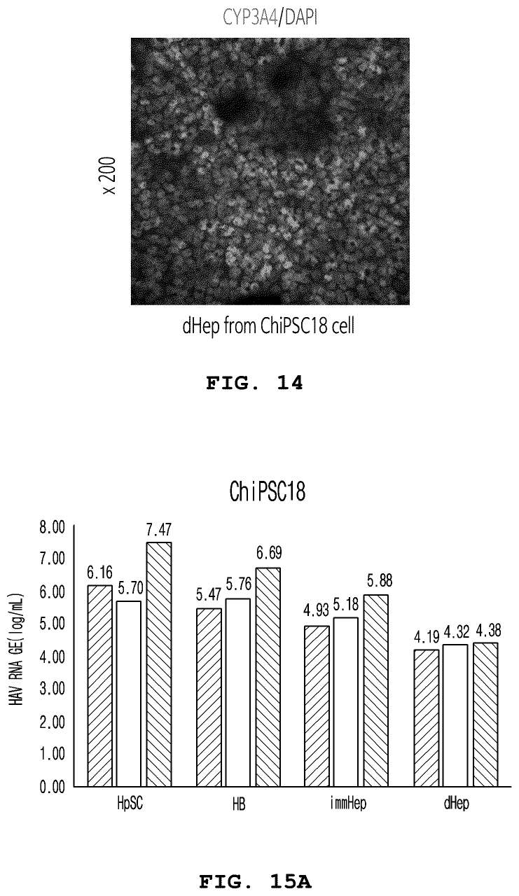

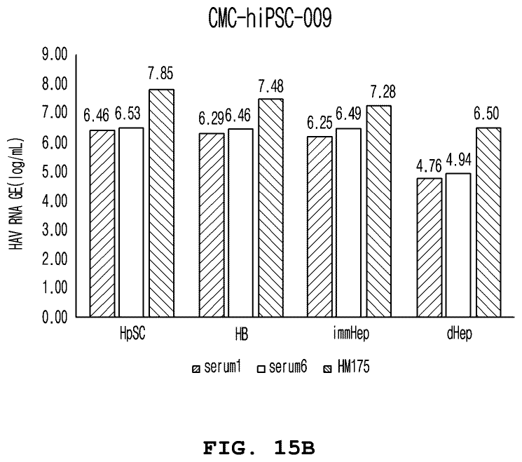

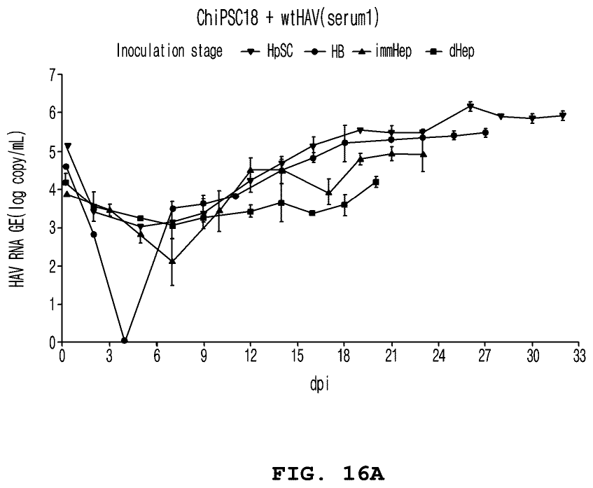

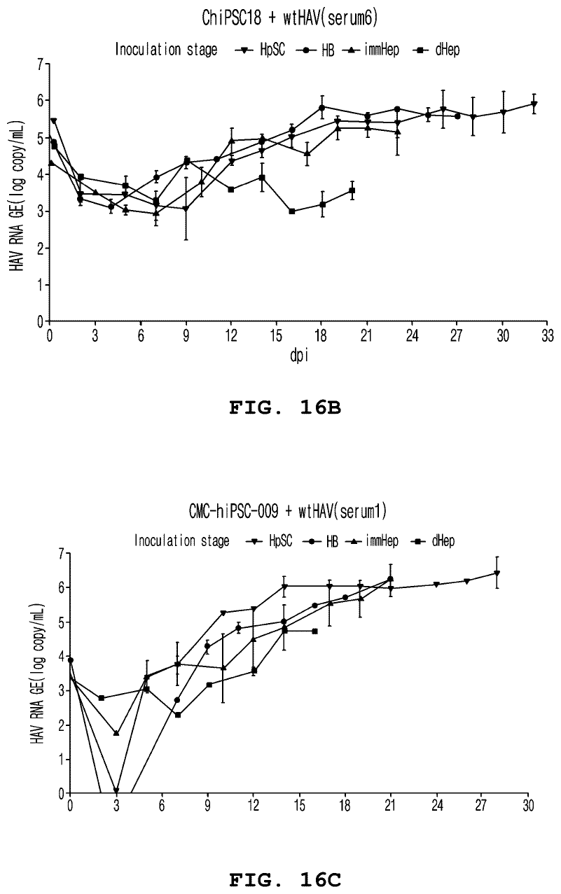

As a result of differentiating iPSCs into hepatocytes, differentiation to DE took a total of 7 days. As shown in FIGS. 9 and 10, it was confirmed that cell morphological characteristics such as tight cellular packing and prominent nucleoli were changed according to DE differentiation. DE cell differentiation was confirmed using IF assay for SOX 17, an DE differentiation marker. As a result, DE differentiation was confirmed by observing the green fluorescence of the DE cell target marker SOX17 (FIGS. 11 and 12).

### <2-4> Confirmation of Hepatocyte Differentiation and Stepwise Differentiation

Hepatocyte differentiation was performed using the Cellartis iPSC to Hepatocyte Differentiation System (Cellartis, Cat #. Y30055). The hepatocyte differentiation method used in the present invention is optimized for 2D culture, and 96 or 24 well plate culture is recommended. The differentiation medium was used at a concentration of 0.5 mL/cm². After removing the medium, observation was performed under a microscope. To prevent the cells from drying out, observation was performed within 1 to 2 minutes after removing the medium. The materials used for hepatocyte differentiation are shown in Table 6, and the hepatocyte differentiation was performed using the following method.

TABLE 6

Materials in Cellartis iPSC to Hepatocyte Differentiation System
1 bottle Hepatocyte Thawing and Seeding Medium (1) (25 mL)
1 bottle Hepatocyte Progenitor Medium (2) (50 mL)
2 bottles Hepatocyte Maturation Medium Base (3A) (24 mL)
1 tube Hepatocyte Maturation Medium Supplement (3B) (2.6 mL)
3 bottles Hepatocyte Maintenance Medium (4) (50 mL)
1 tube Hepatocyte Coating (7.5 mL)

Additional materials required for culture
Fetal bovine serum (FBS) or KnockOut Serum Replacement (KO-SR)
PBS Dulbecco's with Ca²+ & Mg²+ (D-PBS +/+)
PBS Dulbecco's w/o Ca²+ & Mg²+ (D-PBS −/−)
TrypLE Select Enzyme (1X), without phenol red
Equipment to check or control the temperature at 16-18° C., such as a water bath, a
Biosafety Cabinet with Temp-Zone area or an infrared thermometer.
Cellartis Hepatocyte Maintenance Medium (Cat. No. Y30051)*
96-well plates, flat bottom, cell-culture treated
24-well plates, flat bottom, cell-culture treated
Cell culture vessels, tissue-culture treated polystyrene surface
General cell culture equipment used in cell culture laboratory
*If prolonged culture (more than 32 days from start of differentiation) is desired

### 1. Day 7: Dissociation and Seeding of Fresh DE Cells

On the day of use, hepatocyte thawing and seeding medium and hepatocyte coating solution were thawed at 2 to 8° C. Hepatocyte coating was applied at 0.15 mL/cm² depending on the area, and all surfaces were coated. Incubation was performed at room temperature for at least 30 minutes. The coating was removed just before seeding and the coating surface was not allowed to dry. TrypLE Select Enzyme (1×) and D-PBS−/− were warmed at 37° C. An appropriate volume of D-PBS−/− containing 10% FBS or KO-SR was warmed at 37° C.

The endoderm culture medium was carefully removed using a manual pipette. DE cells were washed by adding warmed D-PBS−/−(0.1 mL/cm²). D-PBS was carefully removed using a manual pipette. After adding warmed TrypLE Select Enzyme (0.1 mL/cm²) to DE cells, the cells were reacted in an incubator for 3 to 5 minutes to dissociate the DE cells. The cell suspension was transferred to a 50 mL tube, and the cell culture vessel was washed by adding 0.1 mL/cm² of 10% FBS or KO-SR in D-PBS−/−. This was transferred to the 50 mL tube using a pipette to dilute the cell suspension 1:1. Count viable cells using a hemocytometer or cell counter with trypan blue staining. Transfer the required volume of cell suspension for seeding at a density of 1.2-1.3×10⁵ cells/cm². Centrifuge the cell suspension at 300×g for 5 minutes at room temperature, then resuspend the pellet in Hepatocyte Thawing and Seeding Medium (1) to a final concentration of 2.6×10⁵ cells/mL. Seed the cell suspension at 1.2-1.3×10⁵ cells/cm², ensuring the correct seeding density for successful hepatic differentiation. Incubate the cell culture vessels at 37° C.±1° C. with 5% CO₂ and ≥90% humidity, leaving them undisturbed overnight. Perform the first medium change on day two after seeding.

2. Days 9 and 11: Changing Differentiation Medium (DE Differentiates into HpSC)

On days 9 and 11 of differentiation, hepatocyte progenitor medium (2) was used. The thawed medium could be stored at 2 to 8° C. for up to 2 weeks, and any remaining medium did not be refrozen. The hepatocyte progenitor medium (2) was thawed at room temperature and divided into appropriate amounts for use. The remaining medium was stored at 2 to 8° C. and used on day 11.

The hepatocyte progenitor medium (2) was warmed at 37° C., and the culture medium in the cell culture vessel was carefully removed using a manual pipette. After carefully adding 0.5 mL/cm² of medium along the wall of the culture vessel, and cultured in a 37° C., 5% CO₂ incubator.

3. Days 14 and 16: Changing Differentiation Medium (HpSC Differentiate into HB)

On days 14 and 16 of differentiation, Hepatocyte Maturation Medium was prepared by adding Hepatocyte Maturation Medium Supplement (3B) to Hepatocyte Maturation Medium Base (3A). The Hepatocyte Maturation Medium Supplement (3B) could be stored at 2 to 8° C. for up to 3 days, and the thawed Hepatocyte Maturation Medium Supplement (3B) should not be refrozen.

On day 14, the Hepatocyte Maturation Medium Supplement (3B) was maintained at 2 to 8° C. The Hepatocyte Maturation Medium Base (3A) was maintained at 16-18° C. until day 16, and 1.3 mL of the Maturation Medium Supplement (3B) (2-8° C.) was mixed with 25 mL of the Hepatocyte Maturation Medium Base (3A) (16-18° C.) to prepare Hepatocyte Maturation medium.

On day 14, the culture medium was carefully removed using a manual pipette, and the mixed Hepatocyte Maturation medium (16-18° C.) was carefully added at a concentration of 0.5 mL/cm², followed by culture in a 37° C., 5% CO₂ incubator. The cells were not moved until day 15 to allow the gelatinized layer to form stably.

On day 16, the medium was replaced in the same manner as on day 14 using a mixture of Hepatocyte Maturation Medium (3A) and (3B), as shown in FIG. 3. The Hepatocyte Maturation Medium, maintained at 16-18° C., was carefully added along the well wall at a volume of 0.5 mL/cm². The cells were then incubated at 37° C. with 5% CO₂. To ensure stable formation of the gelatinized layer, the cells remained undisturbed until day 17.

4. Changing Differentiation Medium after Day 18 (HB Differentiates into dHep)

Thawed Hepatocyte Maintenance Medium (4) should be stored at 2-8° C., and it is recommended to be used up within 3 days. Refreezing is not recommended.

The Hepatocyte Maintenance Medium (4) was thawed at room temperature, and the remaining Hepatocyte Maintenance Medium (4) was stored at 2 to 8° C. As shown in FIG. 3, the Hepatocyte Maintenance Medium (4) (37° C.) at a concentration of 0.5 mL/cm² was carefully replaced on the well wall, followed by culture in a 37° C., 5% CO₂ incubator.

For differentiation analysis of hepatocyte differentiation stages, the IF assay was used. The antibodies used herein are shown in Table 7.

TABLE 7

| Antibody | Differentiation stages and roles | Host | Clonality |
|---|---|---|---|
| EpCAM | hepatic stem cells:epithelial | mouse | Monoclonal |
| | cell adhesion molecule | | |
| HNF4A | hepatic stem cells:hepatocyte | mouse | Monoclonal |
| | nuclear factor | | |
| AFP | hepatoblasts:immature | rabbit | Polyclonal |
| | hepatocyte | | |
| CYP3A4 | hepatocytes:cytochrome | mouse | Monoclonal |
| | P450 3A4 | | |

(differentiation markers for each hepatocyte differentiation stage)

As shown in FIG. 1, hepatocytes were differentiated through the following four stages: HpSC, HB, immHep, and dHep). Differentiation from DE cells into HpSCs cells took 6 to 7 days, differentiation from HpSCs into HBs took 5 to 6 days, differentiation from HB into immHep took 4 to 5 days, and differentiation from immHep into dHep took about 10 days. However, according to the literature, the cells can be used for desired applications from day 21 (based on day 0 of undifferentiated iPSC seeding). It is known that dHep could be maintained for at least 32 days when cultured according to the Cellartis iPSC to Hepatocyte Differentiation system (Cellartis, Cat #. Y30055), and that it can be performed from day 23 when high CYP activity is required.

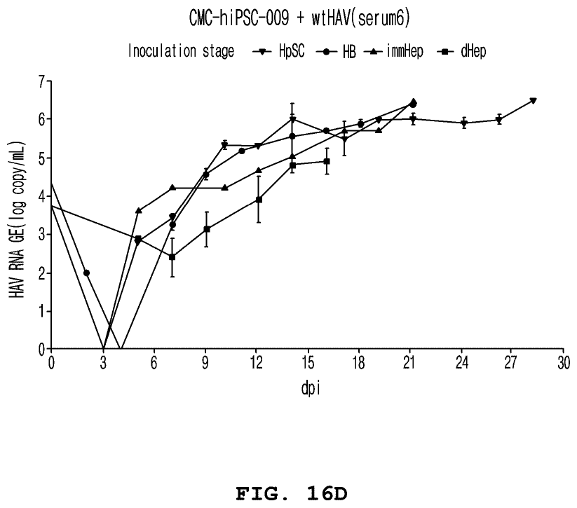

The results of observing the cell morphology at each differentiation stage are shown in FIG. 13, which shows the morphological changes in ChiPSC18, CMC-hiPSC-009 iPSCs at each differentiation stage (HpSC, HB, immHep, and dHep). HpSC were oval with large nuclei and little cytoplasm, while HB and immHep were found to have changed into polygonal cells with an intermediate size between HpSC and hepatocytes (FIG. 13). ChiPSC18 cell activity was confirmed using the CYP3A4 hepatocyte marker. As a result, ChiPSC18 cells with hepatocyte differentiation were confirmed by observing green fluorescence (FIG. 14).

### Example 3: Culture of wtHAV in Differentiated Hepatocytes

The optimal culture time point was explored by inoculating wtHAV at each of the hepatocyte differentiation stages, HpSC, HB, immHep, and dHep (FIG. 4). Two types of wtHAVs were inoculated and cultured at each differentiation stage. Both wtHAVs were genotype IA, and the serum of hepatitis A patients was used as inoculum. The inoculation genome copy number dose was 4.00 log₁₀/mL.

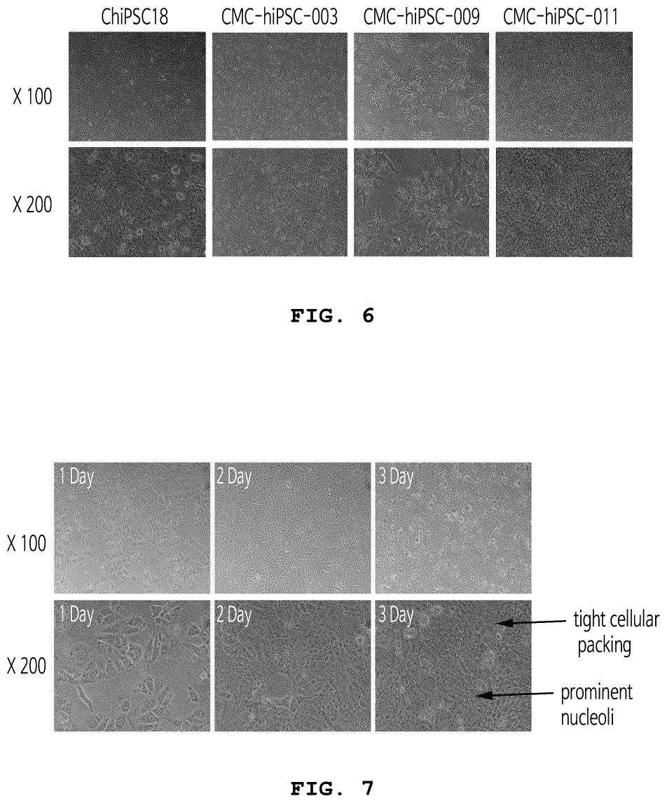

All inoculum was attached 1 hour after inoculation. After recovering the entire culture medium at each detection time point, fresh medium containing differentiation signal molecules was added to simultaneously culture wtHAV and differentiate hepatocytes (Limit of Detection, LOD: 2.5 RNA genome copies log₁₀/mL). The experiment was conducted as shown in FIG. 5. RNA was extracted from the culture medium recovered on each day, and the genome copy number was confirmed using one-step RT-qPCR.

The maximum replication amount at each differentiation stage was confirmed after HAV inoculation.

When ChiPSC18 cells were inoculated with wtHAV serum 1 at different stages of hepatocyte differentiation, the number of wtHAV replicated RNA copies was 6.16 log₁₀/mL in HpSC, 5.47 log₁₀/mL in HB, 4.93 log₁₀/mL in immHep, and 4.19 log₁₀/mL in dHep. On the other hand, when inoculated with wtHAV serum 6 (wtHAV serum 6), the number was 5.70 log₁₀/mL in HpSC, 5.76 log₁₀/mL in HB, 5.18 log₁₀/mL in immHep, and 4.32 log₁₀/mL in dHep.

When CMC-hiPSC-009 cells were inoculated with wtHAV serum 1 at different stages of hepatocyte differentiation, the number of wtHAV replicated RNA copies was 6.48 log₁₀/mL in HpSC, 6.29 log₁₀/mL in HB, 6.25 log₁₀/mL in immHep, and 4.76 log₁₀/mL in dHep. On the other hand, when inoculated with wtHAV serum 6, the number was 6.53 log₁₀/mL in HpSC, 6.48 log₁₀/mL in HB, 6.49 log₁₀/mL in immHep, and 4.94 log₁₀/mL in dHep. The reproducibility of the culture was also confirmed.

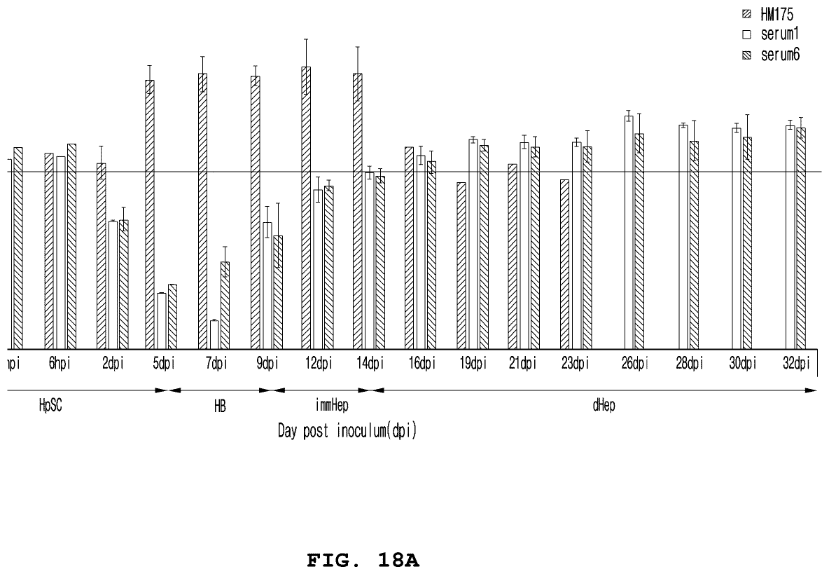

When HAV HM175 strain, used as a positive control, was inoculated into ChiPSC18 cells, the number of wtHAV replicated RNA copies was 7.47 log₁₀/mL in HpSC, 6.69 log₁₀/mL in HB, 5.88 log₁₀/mL in immHep, and 4.38 log₁₀/mL in dHep. When the HM175 strain was inoculated into CMC-hiPSC-009 cells, the number was 7.85 log₁₀/mL in HpSC, 7.48 log₁₀/mL in HB, 7.28 log₁₀/mL in immHep, and 6.50 log₁₀/mL in dHep (FIG. 15).

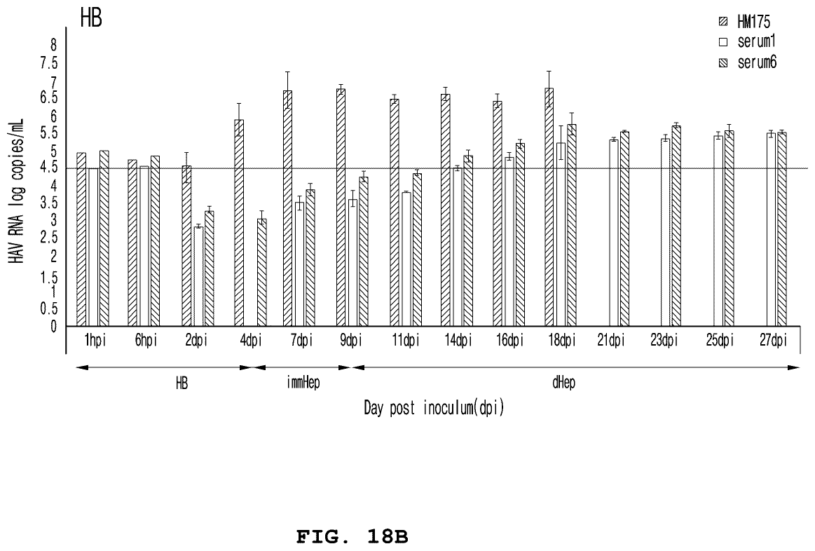

The culture results for each differentiation stage were confirmed over time after HAV inoculation (FIGS. 16A to 16D). As a result, the LOD of this culture method was 2.5 log₁₀/mL, which showed a tendency to decrease after 0 dpi (wtHAV inoculation), but showed a tendency to increase in the wtHAV gene copy number after 3 dpi. These results indicate that the hepatocytes differentiated from iPSCs can function as a sufficient suspect host for wtHAV culture.

In particular, it was confirmed that 5.70 to 6.53 log₁₀/mL of HAV replicated in HpSC and 5.47 to 6.46 log₁₀/mL of HAV replicated in HB when differentiated into hepatocytes after inoculation in the early stage of differentiation (HpSC, HB), and the culture reproducibility was also confirmed. This result was in contrast to previous reports of HAV replicating in hepatocytes, and wtHAV was not cultured in differentiated dHep.

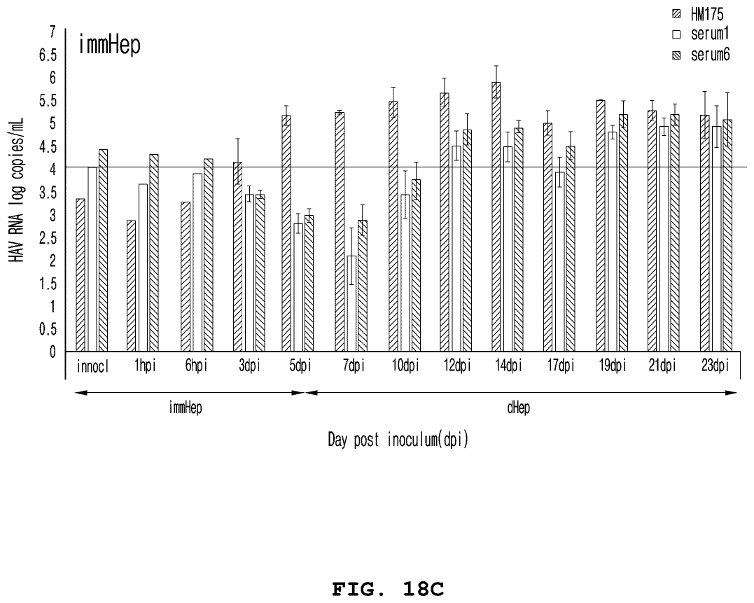

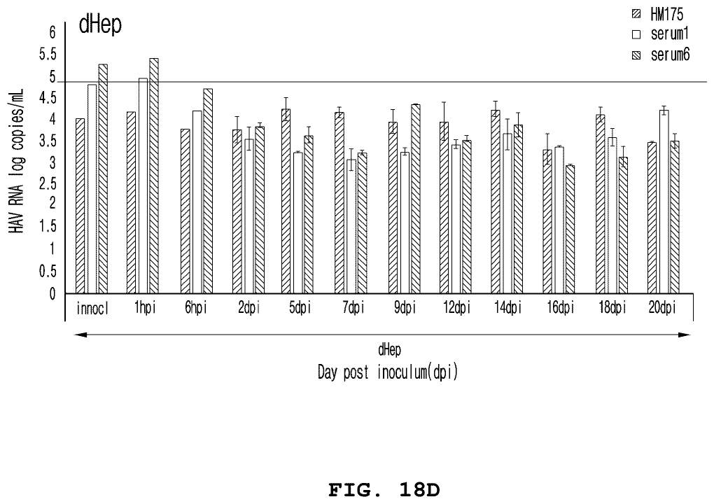

wtHAV was inoculated into HpSCs and observed by performing an IF assay. The detection of HAV capsid protein (VP3) in HpSC by repeating serum 1 and serum 6 from hepatitis A patients twice confirmed that wtHAV was cultured in HpSC because green fluorescence was observed (FIG. 17). The culture results for each wtHAV strain were confirmed according to the culture time of wtHAV. As a result, when ChiPSC18, an iPSC derived from skin fibroblasts, was inoculated with wtHAV genotype IA, serum1 and serum6 were cultured to more than 6 log₁₀/mL after 14 days of culture, and HAV genotype IB, FRhK4-adapted strain (HM175), was cultured to more than 7 log₁₀/mL after 7 days of culture (FIGS. 18A to 18D).

The culture results of HAV (HM175 strain) according to hepatocyte differentiation stage are as shown in FIG. 19. When HM175 was inoculated at 4.60 log₁₀/mL in the HpSC stage, it was cultured at 7.12 log₁₀/mL after 5 dpi, and cell death was observed after the HB lineage. When HM175 was inoculated at 3.52 log₁₀/mL in the HB stage, HAV was cultured steadily as differentiation progressed, reaching a peak of 6.80 log₁₀/mL at 9 dpi. In the immHep stage, HAV was cultured at 5.35 log₁₀/mL only 5 dpi after inoculation with HM175 at 3.59 log₁₀/mL. A trend to increase the HAV replication amount was observed depending on the lineage change point of differentiation.

While the conventional wtHAV culture method (Wheeler, C. M., et al. (1986)) takes more than 4 weeks to culture wtHAV, this culture method was confirmed to shorten the culture period and establish an efficient culture system by inoculating hepatitis viruses by differentiation stage and selecting the optimal time of culture.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The use of the terms “a” and “an” and “the” and “at least one” and similar referents in the context of describing the invention (especially in the context of the following claims) are to be construed to cover both the singular and the plural, unless otherwise indicated herein or clearly contradicted by context.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A method of culturing wild-type hepatitis A virus (wtHAV) using induced differentiated pluripotent stem cell (iPSC)-derived hepatocytes (dHep), comprising the following steps:
a) a step of inoculating wtHAV within a set period of time according to the cell differentiation stage before the iPSCs differentiate into dHEP; and
b) a step of culturing for a set period of time according to the cell differentiation stage after inoculation in step a).

2. The method of culturing wtHAV according to claim 1, wherein the cell differentiation stage is selected from the group consisting of a hepatocyte progenitor (HpSC) differentiation stage, a hepatoblast (HB) differentiation stage, and an immature hepatocyte (immHep) differentiation stage.

3. The method of culturing wtHAV according to claim 1, wherein the iPSCs are selected from the group consisting of ChiPSC18 cells, CMC-hiPSC-003 cells, CMC-hiPSC-009 cells, and CMC-hiPSC-011 cells.

4. The method of culturing wtHAV according to claim 1, wherein the inoculation of the wtHAV is inoculation of serum, blood, sputum, urine, or biological tissue taken from a patient infected with HAV, or food contaminated with HAV.

5. The method of culturing wtHAV according to claim 1, wherein the genotype of the wtHAV is IA, IB, IIA, IIB, IIIA or IIIB.

6. The method of culturing wtHAV according to claim 1, wherein if the inoculation of the wtHAV is performed in the hepatocyte progenitor differentiation stage, the set period of time is between 7 and 13 days from the initial culture time, in which iPSCs have undergone definitive DE and differentiated into HpSC.

7. The method of culturing wtHAV according to claim 6, wherein the inoculation of the wtHAV is performed on day 9 from the initial culture time.

8. The method of culturing wtHAV according to claim 1, wherein if the inoculation of the wtHAV is performed in the HB differentiation stage, the set period of time is between 13 and 18 days from the initial culture time, in which iPSCs have undergone definitive DE and HpSC, and differentiated into HB.

9. The method of culturing wtHAV according to claim 8, wherein the inoculation of the wtHAV is performed on day 14 from the initial culture time.

10. The method of culturing wtHAV according to claim 1, wherein if the inoculation of the wtHAV is performed in the immHep differentiation stage, the set period of time is between 18 and 21 days from the initial culture time, in which iPSCs have undergone DE, HpSC and HB, and differentiated into immHep.

11. The method of culturing wtHAV according to claim 10, wherein the inoculation of the wtHAV is performed on day 18 from the initial culture time.

12. The method of culturing wtHAV according to claim 1, wherein if the inoculation of the wtHAV is performed in the HpSC differentiation stage, the set period of time of step b) is between 14 and 32 days.

13. The method of culturing wtHAV according to claim 1, wherein if the inoculation of the wtHAV is performed in the HB differentiation stage, the set period of time of step b) is between 14 and 27 days.

14. The method of culturing wtHAV according to claim 1, wherein if the inoculation of the wtHAV is performed in the immHep differentiation stage, the set period of time of step b) is between 12 and 23 days.

15. The method of culturing wtHAV according to claim 1, wherein the culture method produces 1.1 to 2.0 times the initial inoculation amount of the wtHAV.
